(19) United States
(12) Patent Application Publication
(10) Pub. No.: US 2015/0025359 A1
(43) Pub. Date: Jan. 22, 2015

(54) METHOD FOR EVALUATION AND COMPARISON OF A CHRONOLOGICAL SEQUENCE OF COMBINED MEDICAL IMAGING EXAMINATIONS AND ALSO A MEDICAL IMAGING SYSTEM WHICH IS DESIGNED FOR EXECUTING THE INVENTIVE METHOD

(71) Applicant: Siemens Aktiengesellschaft, Munich (DE)

(72) Inventors: Matthias FENCHEL, Erlangen (DE); Björn HEISMANN, Erlangen (DE); Kirstin JATTKE, Nuernberg (DE); Ralf LADEBECK, Erlangen (DE); Sebastian SCHMIDT, Weisendorf (DE)

(21) Appl. No.: 14/321,858

(22) Filed: Jul. 2, 2014

(30) Foreign Application Priority Data
Jul. 17, 2013 (DE) 102013214023.1

(51) Int. Cl. G01R 33/48; A61B 5/00

(52) U.S. Cl. CPC G01R 33/481; A61B 5/0035
USPC 600/411

(57) ABSTRACT

A method is disclosed for evaluating and comparing a chronological sequence of at least two combined medical imaging examinations, wherein a combined medical imaging examination is carried out in each case by way of a first imaging apparatus and by way of a second imaging apparatus which is formed by a Positron Emission Tomography apparatus. On the basis of the image data captured by the first imaging apparatus, a transformation specification is created and this is applied to the image data captured by the Positron Emission Tomography apparatus, so that a direct comparison of the first and second image data captured by way of the Positron Emission Technology apparatus is made possible.

### PRIORITY STATEMENT

The present application hereby claims priority under 35 U.S.C. §119 to German patent application number DE 102013214023.1 filed Jul. 17, 2013, the entire contents of which are hereby incorporated herein by reference.

### FIELD

At least one embodiment of the invention generally relates to medical imaging examinations and/or systems therefor.

### BACKGROUND

In combined imaging systems, especially those combining a magnetic resonance apparatus with a Positron Emission Tomography apparatus, the image data is captured by way of two imaging methods simultaneously or directly after one another, wherein the two imaging methods are embodied differently in respect of their technology. Two or more image datasets are captured here, which represent the same anatomical structures but have different image contents however, for example in respect of an image contrast and/or in respect of a voxel size.

Combined imaging examinations are frequently carried out at intervals of a number of days or weeks on the patient for presenting individual areas of the patient's body in order to determine a change of a signal intensity over time. For example a decreasing signal intensity shows a decreasing metabolism activity and thus an effectiveness of the therapy, which can be independent of a size of a lesion.

However a comparison of the examinations and/or image data which has been captured at different points in time is especially difficult since the observed areas of the body, especially lesions, can change their shape and position as a result of different influences. These influences can for example be a nutritional state of the patient and/or a general state of the patient and/or an operation which has been carried out in the interim and/or a support of the patient and/or a change in size of the lesions under observation. This makes it difficult however to assign the individual areas of the body, especially lesions for a time-dependent evaluation.

US 2010 0254 584 discloses the evaluation of a chronological series of magnetic resonance image datasets. Here the individual datasets are registered with one another, segmented and subsequently evaluated. However such an evaluation cannot be transferred to Positron Emission Tomography image data, since in the Positron Emission Tomography image data an anatomical representation is insufficient. In addition individual regions and/or areas can only be delineated to from one another to an insufficient extent in Positron Emission Tomography as a result of a low edge sharpness and/or a poor signal-to-noise ratio.

### SUMMARY

At least one embodiment of the present invention is directed to an evaluation and/or a comparison of a chronological sequence of Positron Emission Tomography image datasets. Advantageous embodiments are described in the dependent claims.

At least one embodiment of the invention is based on a method for an evaluation and comparison of a chronological sequence of at least two combined medical imaging examinations, wherein a combined medical imaging examination is carried out in each case by way of a first imaging apparatus and by way of the second imaging apparatus which is formed by a Positron Emission Tomography apparatus, comprising:
Provision of first image data captured by way of the first imaging apparatus and first image data captured by way of the Positron Emission Tomography apparatus of the first imaging examination,
Provision of second image data captured by way of the first imaging apparatus and second image data captured by way of the Positron Emission Tomography apparatus of the second imaging examination, wherein a period of at least one day has elapsed between the first imaging examination and the second imaging examination,
Creation of a transformation specification on the basis of the first and second image data captured by way of the first imaging apparatus,
Application of the transformation specification to the second image data captured by way of the Positron Emission Tomography apparatus,
Segmentation of the first and/or second image data captured by way of the Positron Emission Tomography apparatus,
Determination of a signal change between the segmented and/or transformed first and second image data captured by way of the Positron Emission Tomography apparatus.

At least one embodiment of the invention is further based on a medical imaging system with a first imaging apparatus, a Positron Emission Tomography apparatus, and a processing unit, wherein the medical imaging apparatus is designed for execution of the method for evaluating and comparing a chronological sequence of at least two combined medical imaging examinations, wherein in each case a combined medical imaging examination is undertaken by way of a first imaging apparatus and by way of a second imaging apparatus which is formed by a Positron Emission Tomography apparatus (20), comprising:
Provision of first image data captured by way of the first imaging apparatus and first image data captured by way of the Positron Emission Tomography apparatus (20) of the first imaging examination,
Provision of second image data captured by way of the first imaging apparatus and second image data captured by way of the Positron Emission Tomography apparatus (20) of the second imaging examination, wherein a period of at least one day has elapsed between the first imaging examination and the second imaging examination,
Creation of a transformation specification on the basis of the first and second image data captured by way of the first imaging apparatus,
Application of the transformation specification to the second image data captured by way of the Positron Emission Tomography apparatus (20),
Segmentation of the first and/or second image data captured by way of the Positron Emission Tomography apparatus (20),
Determination of a signal change between the segmented and/or transformed first and second image data captured by way of the Positron Emission Tomography apparatus (20).

At least one embodiment of the invention is further based on a computer program which is able to be loaded directly into a memory of a programmable processing unit of the medical imaging system, with program segments/modules to execute a method for evaluation and comparison of a chronological sequence of at least two combined medical examinations when the computer program is executed in the processing unit of the medical imaging system. Such a software realization has the advantage that existing processing units of medical imaging devices having a Positron Emission Tomography apparatus can be modified in a suitable manner by implementing the computer program in order in the inventive manner of at least one embodiment to evaluate and compare a chronological sequence of at least two combined medical imaging examinations.

### BRIEF DESCRIPTION OF THE DRAWINGS

Further advantages, features and details of the invention emerge from the example embodiments described below and also with reference to the drawings.

In the figures:

FIG. 1 shows a flowchart of an embodiment of an inventive method for evaluating and comparing a chronological sequence of at least two combined medical imaging examinations and

FIG. 2 shows an embodiment of an inventive medical imaging system in a schematic diagram.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will he understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is riot intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

At least one embodiment of the invention is based on a method for an evaluation and comparison of a chronological sequence of at least two combined medical imaging examinations, wherein a combined medical imaging examination is carried out in each case by way of a first imaging apparatus and by way of the second imaging apparatus which is formed by a Positron Emission Tomography apparatus, comprising:
Provision of first image data captured by way of the first imaging apparatus and first image data captured by way of the Positron Emission Tomography apparatus of the first imaging examination,
Provision of second image data captured by way of the first imaging apparatus and second image data captured by way of the Positron Emission Tomography apparatus of the second imaging examination, wherein a period of at least one day has elapsed between the first imaging examination and the second imaging examination,
Creation of a transformation specification on the basis of the first and second image data captured by way of the first imaging apparatus,
Application of the transformation specification to the second image data captured by way of the Positron Emission Tomography apparatus,
Segmentation of the first and/or second image data captured by way of the Positron Emission Tomography apparatus,
Determination of a signal change between the segmented and/or transformed first and second image data captured by way of the Positron Emission Tomography apparatus.

The inventive embodiment especially enables an evaluation over time of different Positron Emission Tomography examinations (PET examinations) which can have been captured at different points in time to be correlated to one another simply and rapidly on a patient. In particular here signal changes in the examined area of the body, especially a lesion area, of the patient can be established especially quickly between the different PET examinations and thus a change in the area of the body examined, especially a lesion and/or its surroundings, during the course of an illness for example and/or a treatment period of a number of days or weeks, can be determined. In particular here changes in a position of the patient between the two medical imaging examinations and/or a change of a lesion form and/or a change of a patient state can be taken into account in a constructively simple manner in the determination of the signal change. A period of several days or weeks has elapsed for example between the first imaging examination and the second imaging examination. During this period the patient can for example undergo treatment, especially a therapy of the lesion area.

In this context a segmentation should especially be understood as a selection of image voxels and/or image areas in the image data, wherein the selected image voxels and/or selected image areas preferably comprise a lesion and/or a lesion area of the patient. The segmentation of the first and/or second image data captured by way of the PET apparatus can here also include a segmentation of the first and/or second image data captured by way of the first imaging apparatus and/or a segmentation of an overlaying of the image data of the first and/or second imaging, wherein a segmentation specification can be determined for this purpose and this can be transferred and/or applied to the first and/or second image data captured by way of the PET apparatus. Furthermore a provision of image data is especially to be understood as a retrieval and/or loading of stored image data from a memory unit and/or a capturing and/or acquisition of raw imaging data by way of the imaging apparatus, wherein the raw imaging data will subsequently be processed into image data.

At least one embodiment of the inventive method can basically be carried out for all combination systems of medical imaging systems appearing sensible to the person skilled in the art, which especially have a PET apparatus in combination with a further imaging apparatus. Especially advantageously however the first imaging apparatus is formed by a magnetic resonance apparatus, since the magnetic resonance image data and the PET image data can be captured simultaneously and thus the captured magnetic resonance image data and the PET image data provide an image of the same volume captured at the same time. In addition image data with a high spatial resolution, especially for presentation of soft tissue, can be provided by way of the magnetic resonance apparatus, which in combination with the PET image data in their turn make possible an exact localization and/or assignment of signals in the PET data to an area of the patient's body.

It is further proposed that the first raw imaging data captured by way of the first imaging apparatus and the first raw imaging data captured by way of the Positron Emission Tomography apparatus be captured simultaneously or shortly after one another, wherein the first image data is determined from the first raw imaging data. In this way, in an especially simple and time-saving manner, the transformation specification and/or a segmentation specification can be transferred and/or exchanged between the first image data captured by way of the Positron Emission Tomography apparatus and the first image data captured by way of the first imaging apparatus, since the captured image data preferably also provide an image of the same volume or the same area of the body of the patient. In addition a simple overlaying of the two sets of first image data captured by means of the Positron Emission Tomography apparatus and by way of the first imaging apparatus can be achieved.

In this context a simultaneous capturing of raw imaging data is especially to be understood as the capturing of the raw imaging data captured by way of the first imaging apparatus and the capturing of the raw imaging data captured by way of the Positron Emission Tomography apparatus of an area of the patient's body being undertaken at least partly concurrently and/or simultaneously, wherein the patient does not undergo any change in their position within a patient examination area of the combined imaging system. Furthermore a short time interval with which the capturing of the first raw imaging data and the capturing of the second raw imaging data follow one another, is especially to be understood as a time interval of a maximum of 15 minutes, advantageously an interval of a maximum of ten minutes and especially advantageously an interval of a maximum of five minutes.

Especially advantageously the first image data captured by way of the first imaging apparatus and the first image data captured by way of the Positron Emission Tomography apparatus are registered jointly. The joint registration of image data especially includes the first image data captured by way of the PET apparatus and the first image data captured by way of the first imaging apparatus capturing an image of the same volume at the same recording time. In the two first sets of image data corresponding structures are imaged at the same position in the first image data. This enables a direct comparison between the two first sets of image data and/or a simple overlaying of the first image data to be achieved.

A similarly simple transmission and/or a simple exchange of a transformation specification and/or a segmentation specification between the second image data captured by way of the PET apparatus and the second image data captured by way of the first imaging apparatus can advantageously be achieved if the second raw imaging data captured by way of the first imaging apparatus and the second raw imaging data captured by way of the Positron Emission Tomography apparatus are captured at the same time or a short time after one another, whereby the second image data is determined from the second raw imaging data.

Especially advantageously there is joint registration of the second image data captured by way of the first imaging apparatus and the second image data captured by way of the Positron Emission Tomography apparatus, so that, for the second image data too, a direct comparison between the two sets of image data and/or a simple overlaying of the second image data can be achieved.

An advantageous capturing a first raw imaging data and/or of second raw imaging data by way of the Positron Emission Tomography apparatus can be achieved if the first raw imaging data captured by way of the Positron Emission Tomography apparatus and/or second raw imaging data is captured during and/or after a radiopharmaceutical has been administered. Preferably the radiopharmaceutical is a radiopharmaceutical suitable for use in oncology, such as 18-FDG (18-Fluordesoxyglucose) and/or 18-FLT (Fluorothymidine) and/or 18-F-L-Tyrosine and/or 11-C-Methionine and/or 11-C-Choline and/or 18-F-Choline and/or further radiopharmaceuticals appearing sensible to the person skilled in the art. The capturing of the raw imaging data begins approximately 30 minutes to 90 minutes after the administration of the radiopharmaceutical.

Especially advantageously the radiopharmaceutical administered before and/or during the capturing of the first raw imaging data captured by way of Positron Emission Tomography is the same as a radiopharmaceutical administered before and/or during the capturing of the second raw imaging data captured by way of the Positron Emission Tomography apparatus. In this way an advantageous comparison between the first image data captured by way of the Positron Emission Tomography apparatus and the second image data captured by way of the Positron Emission Tomography apparatus can be achieved during an evaluation of the image data and thus the course of an illness and/or progress of the therapy can be advantageously presented.

In a further embodiment of the invention it is proposed that the transformation specification comprises a shift and/or a scaling of individual voxels with a same section of the patient between the first and second image data captured by way of the first imaging apparatus. This advantageously enables a transformation specification to be provided for the evaluation of the PET image data, which takes account of the changed position parameters and/or changed patient parameters. Preferably the shift and/or a scaling of individual voxels comprises a shift and/or scaling in relation to a coordinate system of the first imaging apparatus and/or the combined imaging system, so that the first image data captured by way of the first imaging apparatus and the second image data captured by way of the first imaging apparatus have the same coordinates within the coordinate system of the first imaging apparatus and/or the combined imaging system.

The shift here can comprise a translation shift and/or a rotation shift. Preferably the first and second image data captured by way of the first imaging apparatus have a higher spatial resolution here than a spatial resolution of the first and second image data captured by way of the Positron Emission Tomography apparatus.

In an advantageous development of at least one embodiment of the invention it is proposed that the signal change comprises a change in signal intensity and/or a change of an SUV. In this way a change in the course of an illness and/or of tumor tissue and/or a lesion and/or a diseased area of the body of the patient etc. can be detected especially simply and directly, and on the basis of this signal change an informative diagnosis can be created. Preferably the SUV (standardized uptake value) comprises a physiological quantification of local concentrations of a radioactivity. The SUV quantitatively includes a metabolism of the radiopharmaceutical used for the Positron Emission Tomography examination with the region of the body examined, for example a tumor. Here the SUV essentially represents a radioactivity concentration in relation to an applied radioactivity.

Especially advantageously a signal intensity is corrected by way of a pharmacokinetic model, through which processes which the radiopharmaceutical applied to the patient executes with the body of the patient can also be taken into consideration for determining the signal intensity and/or for establishing the signal change. For example an arterial input function in the take-up of medical substances, especially the radiopharmaceutical, can be taken into account here in the computation and/or determination of the change of signal intensity. Pharmacokinetic models take account in such cases where possible of a totality of all processes which a radiopharmaceutical applied to the patient undergoes in the body of the patient. These processes include processes such as taking up of the radiopharmaceutical, distribution of the radiopharmaceutical in the body, a breakdown of the radiopharmaceutical and a discharge of the radiopharmaceutical.

Furthermore it is proposed that at least one parameter for the correction by way of a pharmacokinetic model is established from the first and/or second image data captured by way of the first imaging apparatus. If the first imaging apparatus is a magnetic resonance apparatus here, an arterial input function, which especially includes an arterial take-up of administered medical substances, can be determined especially advantageously from the first image data and/or the second image data of a magnetic resonance measurement.

At least one embodiment of the invention is further based on a medical imaging system with a first imaging apparatus, a Positron Emission Tomography apparatus, and a processing unit, wherein the medical imaging apparatus is designed for execution of the method for evaluating and comparing a chronological sequence of at least two combined medical imaging examinations, wherein in each case a combined medical imaging examination is undertaken by way of a first imaging apparatus and by way of a second imaging apparatus which is formed by a Positron Emission Tomography apparatus (20), comprising:
Provision of first image data captured by way of the first imaging apparatus and first image data captured by way of the Positron Emission Tomography apparatus (20) of the first imaging examination,
Provision of second image data captured by way of the first imaging apparatus and second image data captured by way of the Positron Emission Tomography apparatus (20) of the second imaging examination, wherein a period of at least one day has elapsed between the first imaging examination and the second imaging examination,
Creation of a transformation specification on the basis of the first and second image data captured by way of the first imaging apparatus,
Application of the transformation specification to the second image data captured by way of the Positron Emission Tomography apparatus (20),
Segmentation of the first and/or second image data captured by way of the Positron Emission Tomography apparatus (20),
Determination of a signal change between the segmented and/or transformed first and second image data captured by way of the Positron Emission Tomography apparatus (20).

Such a medical imaging system makes a temporal evaluation of different PET examinations which were captured at different points in time on a patient in correlation to one another especially easy and fast. Signal changes in a region of the body examined, especially a lesion area of a patient between the different PET examinations, can be established here especially quickly and thus a change in the region of the body examined, especially of the lesion and/or its environment during the course of an illness for example and/or a treatment period of several days or weeks can be determined. In particular changes in a position of the patient between the two medical imaging examinations and/or a change of a form of lesion and/or a change of a patient state can be taken into account in a constructively simple manner here in the determination of the change.

At least one embodiment of the invention is further based on a computer program which is able to be loaded directly into a memory of a programmable processing unit of the medical imaging system, with program segments/modules to execute a method for evaluation and comparison of a chronological sequence of at least two combined medical examinations when the computer program is executed in the processing unit of the medical imaging system. Such a software realization has the advantage that existing processing units of medical imaging devices having a Positron Emission Tomography apparatus can be modified in a suitable manner by implementing the computer program in order in the inventive manner of at least one embodiment to evaluate and compare a chronological sequence of at least two combined medical imaging examinations.

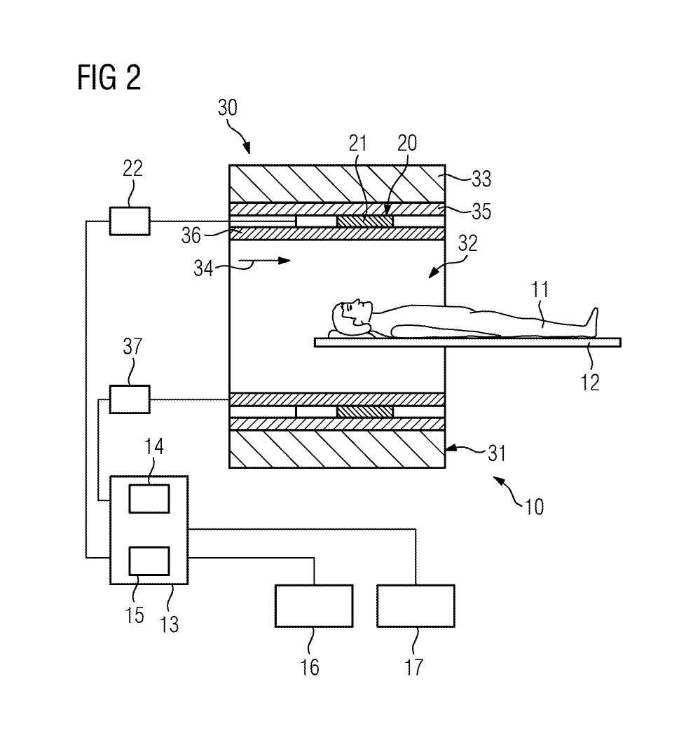

FIG. 2 shows a schematic diagram of the medical imaging system 10. The medical imaging system 10 comprises a combined imaging system having a first imaging apparatus and a second imaging apparatus. The second imaging apparatus is formed by a Positron Emission Tomography apparatus 20 (PET apparatus 20). In the present exemplary embodiment the first imaging apparatus is formed by a magnetic resonance apparatus 30. Fundamentally an embodiment of the first imaging apparatus as a computed tomography apparatus etc. is also conceivable, which is able to be combined with the PET apparatus 30.

The magnetic resonance apparatus 30 of the medical imaging system 10 comprises a magnet unit 31. The magnet unit 31 surrounds a patient receiving area 32 for capturing an image of a patient 11, wherein the patient receiving area 32 is surrounded in a circumferential direction by the magnet unit 31 in a cylindrical shape. The patient 11 can be pushed by way of the patient support apparatus 12 of the medical imaging system 10 into the patient receiving area 32. For this purpose the patient support apparatus 12 is disposed so as to enable it to be moved within the patient receiving area 32.

The magnet unit 31 comprises a main magnet 33, which is designed to create a strong and especially constant main magnetic field 34 during the operation of the magnetic resonance apparatus 30. The magnet unit 31 also has a gradient coil unit 35 for creating magnetic field gradients, which is used for local encoding during imaging. In addition the magnet unit 31 includes a high-frequency antenna unit 36 which is designed to excite a polarization which is generated in the main magnetic field 34 created by the main magnet 33.

To control the main magnet 33 of the gradient coil unit 35 and to control the high-frequency antenna unit 36 the magnetic resonance apparatus 30 has a control unit 37 formed by the processing unit. The control unit 37 centrally controls the magnetic resonance apparatus 30, such as for example the execution of a predetermined imaging gradient echo sequence. For this purpose the control unit 37 comprises a gradient control unit is not shown in any greater detail and a high-frequency antenna control unit is not shown in any greater detail. In addition the control unit 37 comprises an evaluation unit not shown in any greater detail for evaluation of magnetic resonance image data.

The magnetic resonance image apparatus 30 shown can of course include further components which magnetic image apparatuses normally include. A general way in which a magnetic resonance apparatus 30 functions is also known to the person skilled in the art, so that a more detailed description of the general components will be dispensed with here.

The PET apparatus 20 of the medical imaging system 10 comprises a number of Positron Emission Tomography detector modules 21 (PET detector modules 21), which are disposed in the shape of a ring and surround the patient receiving area 32 in the circumferential direction. The PET detector modules 21 are disposed here between the high-frequency antenna unit 36 and the gradient coil unit 35 of the magnetic resonance apparatus 30 and are thus integrated into the magnetic resonance apparatus 30 in an especially space-saving way.

The PET detector modules 21 each have a number of Positron Emission Tomography detector elements (PET detector elements) riot shown in any greater detail, which are arranged to form a PET detector array which comprises a scintillation detector array with scintillation crystals, for example LSO crystals. Furthermore the PET detector module 21 includes a photodiode array in each case, for example an avalanche photodiode array or APD photodiode array, which are disposed downstream from the scintillation detector array within the PET detector module 21. The PET detector array additionally has detector electronics, not shown in any greater detail, which includes an electric amplifier circuit and further electronic components not shown in any greater detail.

To control the PET detector module 21 the PET apparatus 20 has a control unit 22. The PET apparatus 20 shown can of course include further components which PET apparatuses normally include. A general way in which a PET apparatus 20 functions is also known to the person skilled in the art, so that a more detailed description of the general components will be dispensed with here.

Photon pairs which result from the annihilation of a positron with an electron are detected by way of the PET detector module 21. Trajectories of the two photons enclose an angle of 180°. In addition the two photons each have an energy of 511 keV. The positron here is emitted by a radiopharmaceutical, wherein the radiopharmaceutical is administered to the patient 11 via an injection. As they pass through material the photons arising during the annihilation can be absorbed, whereby the absorption probability depends on the path length through the material and the corresponding absorption coefficient of the material.

The medical imaging system 10 also has a central processing unit 13, which for example harmonizes a detection of magnetic resonance image data by way of the magnetic resonance apparatus 30 and the detection of PET image data by way of the PET apparatus to one another for a joint data capture. The processing unit 13 also includes an evaluation unit not shown in any greater detail. The processing unit 13 further includes a processor unit 14 and a memory unit 15. Control information such as imaging parameters for example and also reconstructed image data, can be displayed on a display unit 16, for example on at least one monitor, of the medical imaging system 10 for an operator. In addition the medical imaging system 10 has an input unit 17, by way of which information and/or parameters can be entered during a measurement process by an operator.

The medical imaging system 10 shown can of course include further components which medical imaging systems normally include. A general way in which a medical imaging system 10 functions is also known to the person skilled in the art, so that a more detailed description of the general components will be dispensed with here.

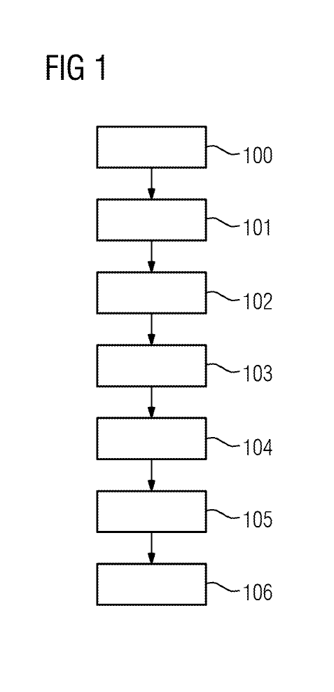

FIG. 1 shows a flowchart of an embodiment of inventive method for evaluating and comparing a chronological sequence of at least two combined medical imaging examinations, wherein a combined medical imaging examination carried out by way of the magnetic resonance apparatus 30 and by way of the PET apparatus 20 in respectively are shown schematically.

In a first method step 100, first image data captured by way of the magnetic resonance apparatus 30, which is formed by first magnetic resonance image data, and first image data captured by way of the PET apparatus 20, which is formed by Positron Emission Tomography image data (PET image data) of a first imaging examination is provided by the processing unit 13 for a further evaluation. The magnetic resonance image data has a higher spatial resolution than the PET image data, so that, by way of the magnetic resonance image data, an especially unique assignment of PET signals in the PET image data to a point of origin in the human body is made possible during an evaluation of the PET image data.

The provision of the first magnetic resonance image data and of the first PET image data in the first method step 100 in this case comprises a retrieval of already stored image data from a memory unit or also a detection and/or acquisition of the first raw imaging data by way of the magnetic resonance apparatus 30 and the PET apparatus 20. The first image data is established here from the first raw imaging data.

The detection of the first raw imaging data provided by way of the PET apparatus 20 occurs during and/or after the administration of a radiopharmaceutical. Preferably the radiopharmaceutical used is a radiopharmaceutical suitable for use in oncology, such as for example 18-FDG (18-Fluordesoxyglucose) and/or 18-FLT (Fluorothymidine) and/or 18-F-L-Tyrosine and/or 11-C-Methionine and/or 11-C-Choline and/or 18-F-Choline and/or further radiopharmaceuticals appearing sensible to the person skilled in the art. The first raw imaging data can start to be captured up to 90 minutes after the administration of the radiopharmaceutical. In addition there can also be provision for the capturing of the first raw imaging data provided by way of the magnetic resonance apparatus 30 to occur during and/or after the administration of a magnetic resonance contrast medium, for example Gd-DTPA.

The capturing of the first raw imaging data provided by way of the magnetic resonance apparatus 30 and the capture of the first raw imaging data provided by way of the PET apparatus 20 is undertaken concurrently or simultaneously. As an alternative to this the provision of the first raw imaging data provided by way of the magnetic resonance apparatus 30 and the provision of the first raw imaging data provided by way of the PET apparatus 20 can also occur consecutively with a short period of time between the two, wherein the short period of time between the capturing of the first raw imaging data by way of the magnetic resonance apparatus 30 and of the first raw imaging data by way of the PET apparatus 20 can be after an interval of a maximum of 15 minutes, preferably of a maximum of ten minutes and especially preferably of a maximum of five minutes.

In addition, in the first method step 100 a joint registration of the first magnetic resonance image data and the first PET image data is undertaken. Through the joint registration the first magnetic resonance image data and the first PET image data map the same volume of a region of the body of the patient 11 captured at the same time. In particular here corresponding structures at a same position are mapped in the first image data.

In a further method step 101, second image data captured by way of the magnetic resonance apparatus 30, which is formed by a second magnetic resonance image data, and second image data captured by way of the PET apparatus 20, which is formed by second PET image data, of a second imaging examination are provided by the processing unit 13. The provision of the first magnetic resonance image data and the first PET image data in the further method step 101 comprises in this case a retrieval already stored, second image data from a memory unit or also a detection and/or acquisition of second raw imaging data by way of the magnetic resonance apparatus 30 and the PET apparatus 20. The second image data is established here from the second raw imaging data.

The capturing of the second image raw data provided by way of the PET apparatus 20 occurs during and/or after the administration of a radiopharmaceutical. Preferably the pharmaceutical used is a pharmaceutical suitable for use in oncology, such as for example 18-FDG (18-Fluordesoxyglucose) and/or 18-FLT (Fluorothymidine) and/or 18-F-L-Tyrosine and/or 11-C-Methionine and/or 11-C-Choline and/or 18-F-Choline and/or further radiopharmaceuticals appearing sensible to the person skilled in the art.

In particular the radiopharmaceutical administered for the capturing of the second raw imaging data provided is embodied the same as the radiopharmaceutical administered for the capturing of the first raw imaging data provided. The capturing of the first raw imaging data by way of the PET apparatus 20 can begin up to 90 minutes after the administration of the radiopharmaceutical, wherein preferably a time sequence of the capturing of the second raw imaging data provided by way of the PET apparatus 20 corresponds essentially to a time sequence of the capturing of the first raw imaging data provided by the PET apparatus 20.

In addition there can also be provision for the capturing of the second raw imaging data provided by way of the magnetic resonance apparatus 30 to be undertaken during and/or after the administration of a magnetic resonance contrast medium, for example Gd-DTPA, wherein here too a time sequence of the capturing of the second raw imaging data provided by way of the magnetic resonance apparatus 30 essentially corresponds to a time sequence of the capturing of the first raw imaging data provided by way of the magnetic resonance apparatus 30.

The capturing of the second raw imaging data provided by way of the magnetic resonance apparatus 30 and the capturing of the second raw imaging data provided by way of the PET apparatus 20 occurs concurrently or simultaneously. As an alternative to this the capturing of the second raw imaging data provided by way of the magnetic resonance apparatus 30 and the capturing of the second raw imaging data provided by way of the PET apparatus 20 can also occur consecutively, with a short period of time between the two, whereby the short period of time between the capturing of the second raw imaging data by way of the magnetic resonance apparatus 30 and the second raw imaging data by way of the PET apparatus 20 has a period of time of maximum 15 minutes, advantageously of maximum ten minutes and especially preferably of maximum of five minutes. In addition, in the second method step 101, a joint registration of the second magnetic resonance image data and the second PET image data is undertaken.

A period of at least one day, preferably however of several days or weeks has passed between the capturing of the first raw imaging data and the capturing of the second raw imaging data, so that by way of a comparison of the first image data and the second image data, especially the course of an illness and/or the course of the therapy which has occurred between the first imaging examination and the second imaging examination, can be presented and/or established.

In a further method step 102, a transformation specification is created by the processing unit 13. The transformation specification is created by the processing unit 13 on the basis of the first magnetic resonance image data and the second magnetic resonance image data, since the magnetic resonance image data has a higher spatial resolution than the PET image data and thus also has a higher accuracy in respect of a presentation and/or an imaging of regions of the body of the patient 11. The transformation specification includes a shift and/or a scaling of the individual voxels of a section of the first or second magnetic resonance image data, so that this data can be compared and/or overlaid with a corresponding section of the second or first magnetic resonance image data. The section of the first magnetic resonance image data and the section of the second magnetic resonance image data in this case capture an image of an identical region of the body of the patient 11.

Here the shift and/or scaling of individual voxels comprises a shift and/or scaling in relation to a coordinate system of the magnetic resonance apparatus 30, so that the first magnetic resonance image data and the second magnetic resonance image data have the same coordinates within the coordinate system of the magnetic resonance apparatus 30. The shift here can comprise a translation shift and/or a rotation shift.

In a further method step 103, the transformation specification is applied to the second PET image data, so that the second PET image data and the first PET image data have the same coordinates within the coordinate system. The further method step 103 is carried out by the processing unit 13.

Following on from this, in a further method step 104, a segmentation of the first PET image data and/or of the second PET image data is carried out. The method step 104 of the segmentation is carried out here by the processing unit 13, wherein a segmentation specification is applied here to first and/or second PET image data already transformed by way of the transformation specification.

As an alternative or in addition the segmentation of the first and/or of the second PET image data here can also comprise a segmentation of the first and/or of the second magnetic resonance image data and/or a segmentation of an overlaying of the first magnetic resonance image data with the first PET image data and/or an overlaying of the second magnetic resonance image data with the second PET image data. Here a segmentation specification can be created by the processing unit 13 on the basis of the segmented first and/or second magnetic resonance image data and/or on the basis of the segmented overlaying of the first magnetic resonance image data with the first PET image data and/or on the basis of the segmented overlaying of the second magnetic resonance image data with the second PET image data. Subsequently in this method step 104, this segmentation specification can be applied by the processing unit 13 to the first and/or second PET image data.

By way of the segmentation and/or the segmentation specification the first and/or the second PET image data is restricted to a relevant image data area, for example to an imaged tumor area and/or to a lesion area etc. of the patient 11.

Subsequently, in a further method step 105, a signal change between the segmented and/or transformed first PET image data and the segmented and/or transformed second PET image data is established by the processing unit 13. The segmented and possibly transformed first and second PET image data here comprise an identical image section of an area of the body of the patient 11 imaged by way of the PET image data, so that by a direct comparison of the first PET image data and the second PET image data, a signal change between the two sets of PET image data and thus a change in the observed and/or presented area of the body is visible.

The signal change here comprises a change of a signal intensity and/or a change of an SUV. In addition, in method step 105, the signal intensity of the first PET image data and/or of the second PET image data can be corrected by way of a pharmacokinetic model. For example an arterial input function for taking up the radiopharmaceutical can be taken into account in the computation and/or determination of the change of signal intensity. For this purpose at least one parameter for the correction by way of the pharmacokinetic model is established by the processing unit 13 and from the first magnetic resonance image data and/or from the second magnetic resonance image data. For example an arterial input function can be determined here from the first magnetic resonance image data and/or the second magnetic resonance image data, which is especially comprises and/or describes an arterial take-up of administered medical substances, for example of the radiopharmaceutical and/or the magnetic resonance contrast medium.

In a method step 106, following said step an optical presentation and/or output is generated by way of the processing unit 13 for the signal change determined and displayed by way of the display unit 16 for a medical operator.

The method steps 100 to 106 are executed by the processing unit 13 together with the magnetic resonance apparatus 30 and the PET apparatus 20. For this purpose processing unit 13 includes the necessary software and/or computer programs which are stored in the memory unit 15. The software and/or computer programs comprise program means which are designed to execute the described method for evaluating and comparing a chronological sequence of at least two combined medical imaging examinations when the computer program and/or the software is executed in the processing unit 13 by way of the processor unit 14 of the medical imaging apparatus 10.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may he stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in greater detail by the preferred example embodiment, the invention is not restricted by the disclosed examples and other variations can be derived therefrom by the person skilled in the art, without departing from the scope of protection of the invention.

What is claimed is:

1. A method for evaluating and comparing a chronological sequence of at least two combined medical imaging examinations, wherein in each case a combined medical imaging examination is carried out by way of a first imaging apparatus and by way of a second imaging apparatus which is formed by a Positron Emission Tomography apparatus, the method comprising:
provisioning first image data captured by the first imaging apparatus and first image data captured by the Positron Emission Tomography apparatus of the first imaging examination;
provisioning second image data captured by the first imaging apparatus and second image data captured by the Positron Emission Tomography apparatus of the second imaging examination, wherein a period of at least one day has elapsed between the first imaging examination and the second imaging examination;
creating a transformation specification based upon the first and second image data captured by the first imaging apparatus;
applying the transformation specification to the second image data captured by the Positron Emission Tomography apparatus;
segmenting at least one of the first and second image data captured by the Positron Emission Tomography apparatus; and
determining a signal change between at least one of the segmented and transformed first and second image data captured by the Positron Emission Tomography apparatus.

2. The method of claim 1, wherein the first imaging apparatus is formed by a magnetic resonance apparatus.

3. The method of claim 1, wherein capturing of first raw imaging data captured by the first imaging apparatus and the capturing of first raw imaging data captured by the Positron Emission Tomography apparatus is undertaken concurrently or consecutively after a short interval, and wherein the first image data is established from the first raw imaging data.

4. The method of claim 1, wherein there is a joint registration of the first image data captured by the first imaging apparatus and the first image data captured by the Positron Emission Tomography apparatus.

5. The method of claim 1, wherein capturing of the first image data captured by the first imaging apparatus and capturing of the first image data captured by the Positron Emission Tomography apparatus is undertaken at the same time, and wherein the second image data is established from the second raw imaging data.

6. The method of claim 1, wherein there is a joint registration of the second image data captured by the first imaging apparatus and the second image data captured by the Positron Emission Tomography apparatus.

7. The method of claim 3, wherein at least one of the first raw imaging data and second raw imaging data captured by the Positron Emission Tomography apparatus is captured at least one of during and after the administration of a radiopharmaceutical.

8. The method of claim 7, wherein the radiopharmaceutical administered at least one of before and during the capturing of the first raw imaging data captured by the Positron Emission Tomography apparatus is identical to a radiopharmaceutical administered at least one of before and during the capturing of the second raw imaging data captured by the Positron Emission Tomography apparatus.

9. The method of claim 1, wherein the transformation specification comprises at least one of a shift and a scaling of individual voxels with an identical section of the patient between the first and second image data captured by the first imaging apparatus.

10. The method of claim 1, wherein the signal change comprises at least one of a change of signal intensity and a change of an SUV.

11. The method of claim 10, wherein the signal intensity is corrected by way of a pharmacokinetic model.

12. The method of claim 1, wherein at least one parameter for the correction by way of a pharmacokinetic model is established from at least one of the first and second image data captured by the first imaging apparatus.

13. A medical imaging system, comprising:
a first imaging apparatus;
a Positron Emission Tomography apparatus; and
a processing unit, wherein the medical imaging apparatus is designed to carry out the method for evaluating and compare at least two combined medical imaging examinations following on from one another in time of claim 1.

14. A computer program, directly loadable into a memory of a programmable processing unit of a medical imaging system, including program segments for executing a method of claim 1 when the computer program is executed in the processing unit of the medical imaging system.

15. The method of claim 5, wherein at least one of the first raw imaging data and second raw imaging data captured by the Positron Emission Tomography apparatus is captured at least one of during and after the administration of a radiopharmaceutical.

16. The method of claim 15, wherein the radiopharmaceutical administered at least one of before and during the capturing of the first raw imaging data captured by the Positron Emission Tomography apparatus is identical to a radiopharmaceutical administered at least one of before and during the capturing of the second raw imaging data captured by the Positron Emission Tomography apparatus.

17. A computer readable medium including program segments for, when executed on a processing unit of a medical imaging system, causing the processing unit of the medical imaging system to implement the method of claim 1.

18. The method of claim 2, wherein capturing of first raw imaging data captured by the first imaging apparatus and the capturing of first raw imaging data captured by the Positron Emission Tomography apparatus is undertaken concurrently or consecutively after a short interval, and wherein the first image data is established from the first raw imaging data.

19. The method of claim 18, wherein there is a joint registration of the first image data captured by the first imaging apparatus and the first image data captured by the Positron Emission Tomography apparatus.

20. The method of claim 4, wherein capturing of the first image data captured by the first imaging apparatus and capturing of the first image data captured by the Positron Emission Tomography apparatus is undertaken at the same time, and wherein the second image data is established from the second raw imaging data.
